(19) United States
(12) Patent Application Publication
(10) Pub. No.: US 2008/0233077 A1
(43) Pub. Date: Sep. 25, 2008

(54) Cosmetic Use of a Particular Copolymer as Skin Tensor in a Cosmetic Composition

(75) Inventor: Guillaume Cassin, Villebon Sur Yvette (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/908,610

(22) PCT Filed: Mar. 17, 2006
(86) PCT No.: PCT/EP2006/060843

§ 371 Date: Sep. 14, 2007

(30) Foreign Application Priority Data
Mar. 17, 2005 (FR) 0550692

(51) Int. Cl. A61K 8/90; A61Q 19/08

(52) U.S. Cl. 424/78.03

(57) ABSTRACT

The present invention relates to the cosmetic use, as a skin-tensioning agent in a cosmetic composition, of a copolymer comprising units derived from styrene and units derived from ethyl (meth)acrylate, in which the weight ratio between the units derived from styrene and the units derived from ethyl (meth)acrylate is greater than or equal to 1.

### TECHNICAL FIELD

The present invention relates to the cosmetic use of a copolymer comprising units derived from styrene and units derived from ethyl (meth)acrylate, as a skin-tensioning agent in a cosmetic composition.

The present invention also relates to a cosmetic process for effacing the wrinkles of wrinkled skin, which consists in applying to the said skin a composition comprising, in a physiologically acceptable medium, at least one copolymer comprising units derived from styrene and units derived from ethyl (meth)acrylate as mentioned above.

The general field of the invention is thus that of ageing of the skin.

In the course of ageing of the skin, different signs appear, reflected especially by a change in the structure and functions of the skin. One of these main signs is the appearance of fine lines and deep wrinkles, the size and number of which increase with age. The skin microrelief becomes less uniform and has an anisotropic nature.

### PRIOR ART

It is common practice to treat these signs of ageing with cosmetic compositions containing active agents capable of combating ageing, such as α-hydroxy acids, β-hydroxy acids and retinoids. These active agents act especially on wrinkles by removing the dead cells from the skin and by accelerating the process of cell renewal. However, these active agents have the drawback of being effective in treating wrinkles only after they have been applied for a certain amount of time, i.e. a time that may be from a few days to several weeks.

Now, the current needs are increasingly tending towards the production of compositions for obtaining an immediate effect, leading rapidly to smoothing-out of the wrinkles and/or fine lines and to the disappearance, even temporarily, of fatigue marks. Such compositions are compositions comprising tensioning agents.

These tensioning agents may especially be polymers of natural or synthetic origin in aqueous dispersion, capable of forming a film that causes shrinkage of the stratum corneum, which corresponds to the superficial horny layer of the epidermis.

Thus, as tensioning agents capable of giving an immediate effect, documents WO 98/29092 [1] and EP 1 038 519 [2] describe aqueous dispersions of a polymer system comprising at least one synthetic polymer. The synthetic polymers mentioned are in the form of a latex or pseudolatex, for example of polycondensate type such as anionic, cationic, nonionic or amphoteric polyurethanes, polyurethane-acrylics, polyurethane-polyvinylpyrrolidones, polyester-polyurethanes, polyether-polyurethanes and polyureas, and mixtures thereof. Latices of grafted silicone polymer are also described, comprising a siloxane portion and a portion consisting of a non-silicone organic chain, one of the two portions constituting the main chain of the polymer and the other being grafted onto the said main chain. Such a grafted silicone polymer (also known as polysilicone-8 according to the CTFA nomenclature) is especially sold by the company 3M under the trade name VS 80.

Document FR 2 828 810 [3] describes compositions with a tensioning effect comprising as tensioning agent a polymer of natural origin consisting of a polysaccharide optionally in the form of a plant extract, in particular an algal extract and a polyhydroxylated moisturizer.

Document FR 2822676 [4] describes skin-smoothing film-forming cosmetic compositions comprising, as film-forming agent, at least one acrylic copolymer whose monomer units are chosen from acrylic acid, methacrylic acid and alkyl (meth)acrylates preferably containing up to 30 carbon atoms.

The anti-wrinkle compositions of the prior art, especially those described above, have the particular feature of often resulting in the appearance of an unattractive lacquered film after applying these compositions to the skin.

There is thus a need for a tensioning agent which, when incorporated into a cosmetic composition, acts such that the resulting composition has efficient mechanical skin-smoothing properties and prevents the appearance of a lacquered film on the skin.

Surprisingly, the Inventors have discovered that by incorporating a particular copolymer into anti-wrinkle compositions, the resulting compositions have the expected properties defined in the above paragraph.

### DESCRIPTION OF THE INVENTION

Thus, according to a first subject, the invention relates to the cosmetic use, as a skin-tensioning agent in a cosmetic composition, of a copolymer comprising units derived from styrene and units derived from ethyl (meth)acrylate, in which the weight ratio between the units derived from styrene and the units derived from ethyl (meth)acrylate is greater than or equal to 1.

According to the invention, it is pointed out that the term “unit derived from styrene” means a unit obtained directly from the styrene monomer by polymerization, i.e. a unit having the following formula:

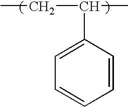

According to the invention, it is pointed out that the term “unit derived from ethyl (meth)acrylate means a unit obtained directly from the ethyl acrylate monomer, in which case the unit corresponds to the following formula:

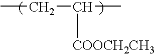

or a unit obtained directly from the ethyl methacrylate monomer, in which case the unit corresponds to the following formula:

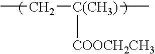

It is pointed out that, in the text hereinabove and hereinbelow, the term “tensioning agent” means a compound capable of having a tensioning effect, i.e. a compound capable of tensioning the skin and, by means of this tensioning effect, smoothing out the skin and causing the wrinkles and fine lines to be reduced or even to disappear immediately.

In addition to having a very efficient tensioning effect, the copolymers of the invention have the particular feature of not giving rise to any unattractive effects, such as the appearance of a glossy lacquered film. They also make it possible to reduce the visibility of the skin's microrelief, thus giving the skin a “soft-focus” effect.

Besides the units derived from styrene and from ethyl (meth)acrylate, the copolymers as defined above may comprise units derived from monomers chosen from (meth)acrylic acid, methyl (meth)acrylate, butyl (meth)acrylate, ethylhexyl (meth)acrylate and 2-hydroxyethyl (meth)acrylate.

The copolymers used according to the invention may be random copolymers, i.e. copolymers comprising a random distribution of the units derived from styrene and derived from ethyl (meth)acrylate, and optionally other units. More specifically, this means that the probability of finding a given monomer unit (styrene, ethyl (meth)acrylate and optionally other units) at any given site in the chain is independent of the adjacent units.

The copolymers used according to the invention may be random copolymers.

Finally, the copolymers used according to the invention may also according to the invention be block copolymers, which constitutes the preferential alternative of the invention.

Advantageously, the block copolymers used according to the invention are linear copolymers containing blocks of the type A-[B-A]ₙ or B-[A-B]ₙ or [A-B]ₙ in which A is a block comprising at least 50% by weight of units derived from styrene, B is a block comprising at least 50% by weight of units derived from ethyl (meth)acrylate, and n is a number greater than or equal to 1.

The blocks A and B link together linearly. If n=1, the copolymer is a triblock copolymer A-B-A or B-A-B, or a diblock copolymer A-B. If n=2, the copolymer is a copolymer A-B-A-B-A, or B-A-B-A-B or A-B-A-B. If n≧3, it is not a star or telechelic copolymer in which a block A or a block B constitutes a core. Preferably, n=1 and more preferably the copolymer is a triblock copolymer A-B-A. It is noted that the copolymer may comprise polymerization functions or groups or residues of such functions or groups, at the end of macromolecular chains. They may be, for example, transfer groups, or residues of transfer groups, for example comprising a group of formula —S—CS—, or a residue of this group.

According to the particular embodiment mentioned above, the block A comprises at least 50% by weight of units derived from styrene, relative to the total weight of the block A. The block A may comprise units other than those derived from styrene, which may be intended to modify the properties of the copolymer or to facilitate its preparation. The block A may thus be a random copolymer comprising units derived from styrene and other units. The solubility of the block A in water or in other media may thus be modified, or its glass transition temperature and thus its rigidity may be modified. The other units of the block A may be units derived from monomers chosen from acrylic acid, methacrylic acid, methyl, butyl, ethylhexyl or 2-hydroxyethyl acrylate, or methyl, butyl, ethylhexyl or 2-hydroxyethyl methacrylate. The presence of small amounts of methacrylic acid may especially facilitate the preparation of the copolymer, the block A may comprise, for example, from 0.1% to less than 5% by weight thereof, relative to the total weight of the block A. The block A preferably comprises at least 75% by weight, preferably at least 90% by weight, and preferably at least 95% by weight of units derived from styrene, relative to the total weight of the block A.

In this case, the other optional unit(s) derived from monomers other than styrene thus preferably represent 25% by weight or less, preferably 10% by weight or less, more preferably 5% by weight or less and even more preferentially about 2% by weight, relative to the total weight of the block A.

The block B as defined above comprises at least 50% by weight of units derived from ethyl acrylate or from ethyl methacrylate. The block B may comprise units other than those derived from ethyl acrylate or from ethyl methacrylate, which may be intended to modify the properties of the copolymer or to facilitate its preparation. The block B may thus be a random copolymer comprising units derived from ethyl acrylate or from ethyl methacrylate and other units. The solubility of the block B in water or in other media may thus be modified, or its glass transition temperature and thus its rigidity may be modified. The other units of the block B may be units derived from monomers chosen from acrylic acid, methacrylic acid, methyl, butyl, ethylhexyl or 2-hydroxyethyl acrylate, and methyl, butyl, ethylhexyl or 2-hydroxyethyl methacrylate. The presence of small amounts of methacrylic acid may especially facilitate the preparation of the copolymer; the block B may, for example, comprise from 0.1% to less than 5% by weight thereof, relative to the total weight of the block. B. Alternatively, the block B may comprise no methacrylic acid-based units. The block B preferably comprises at least 75% by weight, preferably at least 90% by weight and preferably at least 95% by weight of units derived from ethyl acrylate or from ethyl methacrylate, relative to the total weight of the block B. In this case, the other optional unit(s) derived from monomers other than ethyl (meth)acrylate thus preferably represent 25% by weight or less, preferably 10% by weight or less and more preferably 5% by weight or less, relative to the total weight of the block B.

The ratio may be determined by calculating the ratio between the monomers introduced for the preparation of the copolymer, or by calculating the ratio between the average molecular masses of the blocks. Preferably, the ratio between the average molecular masses of the blocks is greater than or equal to 1, preferably greater than or equal to 1.5, preferably greater than or equal to 2.01, preferably greater than or equal to 2.5, and preferably greater than or equal to 5.

It is pointed out that the above-mentioned average molecular masses are theoretical or “targeted” average molecular masses of the block.

The theoretical average molecular mass of a block, M_{block}, is typically calculated according to the following formula:

$${M}_{block} = {\sum }_{i} {M}_{i} * \frac{{n}_{i}}{{n}_{precursor}} ,$$

in which Mᵢ is the molar mass of a monomer i, nᵢ is the number of moles of the monomer i, n_{precursor} is the number of moles of functions to which the macromolecular chain of the block will be attached. The functions may be derived from a transfer agent (or a transfer group) or an initiator, a preceding block, etc. If it is a preceding block, the number of moles may be considered as the number of moles of a compound to which the macromolecular chain of the said preceding block has been attached, for example a transfer agent (or a transfer group) or an initiator. In practice, the theoretical average molecular masses are calculated from the number of moles of monomers introduced and from the number of moles of precursor introduced.

The average molecular mass measured for a first block or for a copolymer denotes the number-average molecular mass as polystyrene equivalents of a block or of a copolymer measured by steric exclusion chromatography (SEC), in THF, with calibration using polystyrene standards. The measured average molecular mass of an n^{th} block in a copolymer containing n blocks is defined as the difference between the measured average molecular mass of the copolymer and the measured average molecular mass of the copolymer containing (n−1) blocks from which it is prepared.

According to the invention, the number-average molecular mass of each block, whether it is a block of a styrene unit or a block of an ethyl acrylate unit, is between 1000 g/mol and 200 000 g/mol and preferably between 5000 g/mol and 100 000 g/mol.

A block copolymer that is particularly advantageous is a triblock copolymer comprising:
a first block comprising units derived from styrene with a number-average molecular mass of 30 000 g/mol;
a second block consisting of units derived from ethyl acrylate with a number-average molecular mass of 10 000 g/mol;
a third block comprising units derived from styrene with a number-average molecular mass of 30 000 g/mol.

A copolymer corresponding to the definition given above may be a copolymer for which the first block and/or the third block, and preferably the first block and the third block, comprise(s), besides the units derived from styrene, units derived from methacrylic acid, for example in a styrene/methacrylic acid mass ratio of 98/2.

A copolymer that is also particularly advantageous is a triblock copolymer comprising:
a first block comprising units derived from styrene with a number-average molecular mass of 32 500 g/mol;
a second block comprising units derived from ethyl acrylate with a number-average molecular mass of 5000 g/mol;
a third block comprising units derived from styrene with a number-average molecular mass of 32 500 g/mol.

Another copolymer that is also particularly advantageous is a triblock copolymer comprising:
a first block comprising units derived from styrene with a number-average molecular mass of 25 000 g/mol;
a second block comprising units derived from ethyl acrylate with a number-average molecular mass of 20 000 g/mol;
a third block comprising units derived from styrene with a number-average molecular mass of 25 000 g/mol.

These triblock copolymers may be in the form of an emulsion in water.

The block copolymers used according to the invention may be obtained via any known method, whether by controlled or non-controlled radical polymerization, ring-opening (especially anionic or cationic) polymerization, anionic or cationic polymerization, or via chemical modification of a polymer.

Living or controlled radical polymerization methods are preferably used, and particularly preferably controlled or living radical polymerization methods using a transfer agent comprising a group of formula —S—CS—, especially known under the names RAFT and MADIX.

Mention may be made, for example, of anionic polymerization and controlled radical polymerization (see “New Method of Polymer Synthesis”, Blackie Academic & Professional, London, 1995, volume 2, page 1, or Trends Polym. Sci. 4, page 183 (1996) by C. J. Hawker), which may be performed according to various processes, for instance atom transfer radical polymerization (or ATRP) (see JACS, 117, page 5614 (1995), from Matyjasezwski et al.)

Using the processes described above, it is possible to prepare a first block starting with monomers or a mixture of monomers, initiators and/or polymerization control agents (transfer agents containing —S—CS— groups, etc.), followed by the growth of a second block on the first block to obtain a diblock copolymer with monomers different from those used for the preparation of the preceding block, and optionally with addition of initiators and/or polymerization control agents, followed by the growth of a third block starting with the diblock copolymer to obtain a triblock copolymer, etc. These processes for preparing block copolymers are known to those skilled in the art. It will be mentioned that the copolymer may have at the end of the chain a transfer group or a residue of a transfer group, for example a group comprising a —S—CS— (for example derived from a xanthate or from a dithioester) or a residue of such a group.

It is mentioned that the triblock copolymers obtained via processes using three successive polymerization blocks are often described as copolymers containing blocks A-B-C. When the composition and the molecular mass of the third block C are substantially identical to the composition and the molecular mass of the first block A (the amounts and proportions of (co)monomers being substantially identical), the triblock copolymers may be described as triblock copolymers A-B-A′ or, by extension or simplification, as triblock copolymers A-B-A.

Thus, for example, a triblock copolymer A-B-A according to the invention may be prepared via a process comprising the following steps:
step a): preparation of the block A by polymerization, preferably controlled radical polymerization, of a composition comprising:
styrene,
a source of free radicals, and
at least one control agent, preferably an agent comprising an —S—CS— group, for example a xanthate or a dithioester,
step b) production of the diblock copolymer A-B, by growth of the block B on the block A, by polymerization, preferably controlled radical polymerization, of a composition comprising:
ethyl acrylate or ethyl methacrylate, and
optionally a source of free radicals,
step c) production of the triblock copolymer A-B-A, by growth of the block A on the diblock A-B, by polymerization, preferably controlled radical polymerization, of a composition comprising:
ethyl acrylate or ethyl methacrylate, and
optionally a source of free radicals,
step d) optionally destruction or deactivation of a group of the control agent.

According to other embodiments, triblock copolymers A-B-A are prepared as two polymerization blocks, using agents comprising two transfer groups or agents comprising one transfer group allowing a transfer at each end of the group, for example a trithiocarbonate comprising a group of formula —S—CS—S—. In such processes, the blocks A are entirely identical, and the block B generally comprises a central group different from the repeating units of the block B. Mention of the presence of a central group in the block B, referred to, for example, as —X—, —X-Z′-X— or R′ hereinbelow, is often omitted for the sake of simplicity.

Thus, triblock copolymers A-B-A may be prepared via the following process:
step a′): preparation of a polymer of formula R-A-X-A-R via polymerization of a composition comprising:
   styrene
   a source of free radicals, and
   at least one control agent of formula R—X—R in which R, which may be identical or different, is an organic group, and X is a transfer group, the agent preferably being a trithiocarbonate comprising a group —X— of formula —S—CS—S— and preferably two identical groups R such as benzyl groups (the agent thus being, for example, dibenzyltrithiocarbonate),
step b′) production of the triblock copolymer R-A-B—X—B-A-R (or more simply A-B-A), via growth of the block B on the blocks A by polymerization, preferably controlled radical polymerization, of a composition comprising:
   ethyl acrylate or ethyl methacrylate, and
   optionally a source of free radicals.

Triblock copolymers A-B-A may also be prepared via the following process:
step a″): preparation of a polymer of formula R-A-X-Z′-X-A-R via polymerization of a composition comprising:
   styrene
   a source of free radicals, and
   at least one control agent of formula R—X-Z′-X—R in which R, which may be identical or different, is an organic group, Z′ is a divalent organic group, and X is a transfer group, preferably a transfer group comprising a group —S—CS—, the said agent being, for example:
   an agent comprising two xanthate groups, in which —X— is a group of formula —S—CS— forming with -Z′- a group of formula —S—CS—O— (xanthate), Z′ being, for example, a group of formula —O—CH₂—CH₂—O—, and R is, for example, a benzyl group or a group of formula H₃C—OOC—CH(CH₃)—, or
   an agent comprising two dithioester groups, in which —X— is a group of formula —S—CS— forming with -Z′- a group of formula —S—CS—C— (dithioester), Z′ being, for example, a phenyl or benzyl group, or a group of formula —CH₂—C₆H₅—CH₂—, and R is, for example, a benzyl group,
step b″): production of the triblock copolymer R-A-B—X-Z′-X—B-A-R (or more simply A-B-A), via growth of the block B on the blocks A by polymerization, preferably controlled radical polymerization, of a composition comprising:
   ethyl acrylate or ethyl methacrylate, and
   optionally a source of free radicals.

Triblock copolymers A-B-A may also be prepared via the following process:
step a′″): preparation of a polymer of formula Z-X—B—R′—B—X-Z via polymerization of a composition comprising:
   ethyl acrylate or ethyl methacrylate, and
   a source of free radicals, and
   at least one control agent of formula Z-X—R′—X-Z in which Z, which may be identical or different, is an organic group, R′ is a divalent organic group, and X is a transfer group, preferably a transfer group comprising a group —S—CS—, the said agent being, for example:
   an agent comprising two xanthate groups, in which —X— is a group of formula —S—CS— forming with Z- a group of formula —O—CS—S-(xanthate), Z- being, for example, an ethoxy group, and —R′— is, for example, a phenyl or benzyl group, or a group of formula —CH₂—C₆H₅—CH₂—,
   an agent comprising two dithioester groups, in which —X— is a group of formula —S—CS— forming with Z- a group of formula C—CS—S— (dithioester), Z- being, for example, a phenyl or benzyl group, and —R′— is, for example, a phenyl or benzyl group, or a group of formula —CH₂—C₆H₅—CH₂—,
step b′″): production of the triblock copolymer Z-X-A-B—R′—B-A-X-Z (or more simply A-B-A), via growth of the block A on the blocks B by polymerization, preferably controlled radical polymerization, of a composition comprising:
   styrene, and
   optionally a source of free radicals,
step d′″): optionally, destruction or deactivation of a group of the control agent.

The polymerizations may be performed in any appropriate physical form, for example in solution in a solvent, in emulsion in water (“latex” process), or in bulk, where appropriate while controlling the temperature and/or the pH so as to make the species liquid and/or soluble or insoluble.

The copolymers used according to the invention, whether they are random, statistical or block copolymers, are advantageously non-elastomeric copolymers.

The term “non-elastomeric copolymer” generally means a copolymer which, when it is subjected to a constraint intended to stretch it (for example by 30% relative to its initial length), does not return to a length substantially identical to its initial length when the constraint ceases.

More specifically, the term “non-elastomeric copolymer” denotes a copolymer with an instantaneous recovery Rᵢ<50% and a delayed recovery R₂ₕ<70% after having been subjected to a 30% elongation. Preferably, Rᵢ is <30% and R₂ₕ<50%. More specifically, the non-elastomeric nature of the copolymer is determined according to the following protocol:

A copolymer film is prepared by pouring a solution of the copolymer in a Teflon-coated mould, followed by drying for 7 days in an environment conditioned at 23±5° C. and 50±10% relative humidity.

A film about 100 μm thick is thus obtained, from which are cut rectangular specimens (for example using a punch) 15 mm wide and 80 mm long.

The samples in the form of specimens are subjected to a tensile stress using a machine sold under the reference Zwick, under the same temperature and humidity conditions as for the drying.

The specimens are pulled at a speed of 50 mm/min and the distance between the jaws is 50 mm, which corresponds to the initial length (l₀) of the specimen.

The instantaneous recovery Rᵢ is determined in the following manner:
the specimen is pulled by 30% (εₘₐₓ), i.e. about 0.3 times its initial length (l₀);
the constraint is released by applying a return speed equal to the pull rate, i.e. 50 mm/min, and the residual elongation of the specimen is measured as a percentage, after returning to zero constraint (εᵢ).

The instantaneous recovery Rᵢ (in %) is determined by the following formula:

Rᵢ=((εₘₐₓεᵢ)/εₘₐₓ)*100

To determine the delayed recovery, the percentage residual elongation of the specimen (ε₂ₕ) two hours after returning to zero constraint is measured.

The delayed recovery R₂ₕ (in %) is given by the following formula:

R₂ₕ=((εₘₐₓ−ε₂ₕ)/εₘₐₓ)*100

As a guide, a copolymer according to one embodiment of the invention has an instantaneous recovery Rᵢ of 10% and a delayed recovery R₂ₕ of 30%.

The Applicant has also demonstrated that, in order for them to be rigid enough to make the skin taut, the copolymers used in the context of the invention, whether they are random, statistical or block copolymers, should have a weight ratio between the units derived from styrene and the units derived from ethyl (meth)acrylate of greater than 1, preferably greater than or equal to 1.5, preferably greater than 2 (especially greater than or equal to 2.01) and preferably greater than or equal to 5. The ratio may also be greater than 20.

The number-average molecular mass of the overall copolymer is generally greater than 10 000 g/mol and preferably greater than 50 000 g/mol. This molecular mass preferably does not exceed 600 000 g/mol.

The mass-average molecular mass of the overall copolymer is generally greater than 20 000 g/mol. It may also be greater than 100 000 g/mol and preferably less than 1 000 000 g/mol.

According to the invention, copolymers described above are used in an anti-wrinkle cosmetic composition.

The copolymers used in the context of the invention are advantageously included, in the anti-wrinkle compositions containing them, in an active material amount ranging from 0.1% to 20% by weight and preferably from 0.5% to 10% by weight.

The term “active material” means the copolymer without solvent and free of the suspension medium resulting from the polymerization process.

It is understood that the said cosmetic composition will comprise, besides the above-mentioned copolymer(s), a physiologically acceptable medium generally suited to topical application to facial skin, i.e. a medium that is compatible with the skin and optionally the eyelashes and the eyebrows.

The said physiologically acceptable medium is generally cosmetically acceptable, i.e. it has a pleasant odour, colour and feel, which are compatible with a cosmetic use, and does not give rise to any unacceptable discomfort (stinging, tautness or redness) liable to put the user off.

The said physiologically acceptable medium generally comprises an aqueous phase.

The compositions in which the specific copolymers of the invention are incorporated may be in any galenical form normally used for topical application, especially in the form of sera (i.e. thickened aqueous solutions) or in the form of emulsions, especially oil-in-water (O/W), water-in-oil (W/O) or multiple (W/O/W or polyol/O/W or O/W/O) emulsions.

When the composition forms an emulsion, it will comprise a fatty phase.

The fatty phase of this composition may consist especially of fatty substances that are liquid at room temperature (25° C. in general) and/or fatty substances that are solid at room temperature, such as waxes, pasty fatty substances and gums, and mixtures thereof. These fatty substances may be of animal, plant, mineral or synthetic origin.

As fatty substances that are liquid at room temperature, often referred to as “oils”, and can be used according to the invention, mention may be made of: hydrocarbon-based oils of animal origin such as perhydrosqualene; hydrocarbon-based plant oils such as liquid triglycerides of fatty acids of 4 to 10 carbon atoms, for instance heptanoic or octanoic acid triglycerides, or sunflower oil, maize oil, soybean oil, grapeseed oil, sesame seed oil, apricot oil, macadamia oil, castor oil, avocado oil, caprylic/capric acid triglycerides, jojoba oil or shea butter; linear or branched hydrocarbons of mineral or synthetic origin, such as isododecane, liquid paraffins and derivatives thereof, petroleum jelly, polydecenes, and hydrogenated polyisobutene such as parleam; synthetic esters and ethers, especially of fatty acids, for instance purcellin oil, isopropyl myristate, 2-ethyl-hexyl palmitate, 2-octyldodecyl stearate, 2-octyl-dodecyl erucate or isostearyl isostearate; hydroxylated esters, for instance isostearyl lactate, octyl hydroxy-stearate, octyldodecyl hydroxystearate, diisostearyl malate, triisocetyl citrate or fatty alkyl heptanoates, octanoates and decanoates; polyol esters, for instance propylene glycol dioctanoate, neopentyl glycol diheptanoate or diethylene glycol diisononanoate; and pentaerythritol esters; fatty alcohols containing from 12 to 26 carbon atoms, for instance octyldodecanol, 2-butyloctanol, 2-hexyldecanol, 2-undecylpentadecanol or oleyl alcohol; partially hydrocarbon-based and/or silicone-based fluoro oils; silicone oils, for instance volatile or non-volatile, linear or cyclic polydimethylsiloxanes (PDMSs) that are liquid or pasty at room temperature, for instance cyclomethicones, dimethicones, optionally comprising a phenyl group, for instance phenyl trimethicones, phenyltrimethylsiloxy-diphenylsiloxanes, diphenylmethyldimethyltrisiloxanes, diphenyl dimethicones, phenyl dimethicones and polymethylphenylsiloxanes; and mixtures thereof.

These oils may be present in a content ranging from 0.01% to 90% and better still from 0.1% to 85% by weight, relative to the total weight of the composition.

The composition according to the invention may also contain ingredients commonly used in cosmetics, such as thickeners; fillers, for instance optionally coated minerals (such as zinc oxide, silica, alumina, boron nitride, talc, sericite or mica); pigments and dyes; sequestrants; fragrances; acidifying or basifying agents; preserving agents; surfactants, and mixtures thereof.

The composition may also contain adjuvants such as clays, starch and derivatives thereof, aqueous dispersions of styrene-acrylic acid copolymers, melamine-formaldehyde or urea-formaldehyde resin particles, aqueous dispersions of polytetrafluoroethylene, vinylpyrrolidone/1-triacontene copolymers, water-dispersible polymers containing units with an LCST, silicone waxes and resins, expanded terpolymer microspheres of vinylidene chloride, acrylonitrile and methacrylate, sold by the company Expancel, Nylon particles, cellulose microbeads and fibres, especially Nylon fibres.

The composition may also contain anti-ageing active agents with an effect complementary to the copolymers defined above, such as at least one compound chosen from keratolytic or prodesquamating agents, for example α-hydroxyacids, β-hydroxyacids, α-ketoacids, β-ketoacids, retinoids and esters thereof, in particular retinyl palmitate and derivatives thereof, such as salicylic acid and derivatives thereof such as 5-n-octanoylsalicylic acid; moisturizers such as polyols; agents for stimulating collagen and/or elastin synthesis or for preventing their degradation; depigmenting or bleaching agents, for instance kojic acid, para-aminophenol derivatives, arbutin and derivatives thereof; anti-glycation agents; agents for stimulating glycoaminoglycan synthesis; dermo-decontracting or muscle-relaxing agents, such as adenosine and magnesium and manganese salts; antioxidants and free-radical scavengers; vitamins, for instance vitamins C, B3 or PP, B5 and E, and derivatives of these vitamins, and especially esters thereof; vitamin K and its derivatives (K1 and K2); ceramides; DHEA and its derivatives and its chemical precursors such as diosgenin and plant extracts containing them (especially Dioscorea plant extracts); coenzyme Q10; and mixtures thereof.

Needless to say, a person skilled in the art will take care to select this or these optional additional compound(s) and/or the amount thereof, such that the advantageous properties of the corresponding composition according to the invention are not, or are not substantially, adversely affected by the envisaged addition.

A person skilled in the art may select the appropriate presentation form, and also the method for preparing it, on the basis of his general knowledge, taking into account firstly the nature of the constituents used, especially their solubility in the support, and secondly the intended use of the composition.

Another subject of the present invention is a cosmetic process for effacing the wrinkles of wrinkled skin, such as the contour of the eyes, comprising a step that consists in applying to the said skin a composition comprising, in a physiologically acceptable medium, at least one copolymer as defined above.

The application is performed according to the usual techniques, for example by applying creams, gels, sera or lotions to the skin intended to be treated, optionally followed by a rinsing step. In the context of this process, the composition may be, for example, a care or cleansing composition, or a makeup composition, in particular a foundation. It is preferably a leave-on composition.

The composition is preferably applied to the face and/or the neck, in particular to the wrinkled areas of the face, and especially around the eyes.

Other characteristics and advantages will emerge more clearly on reading the examples that follow, which are given as non-limiting illustrations.

### DETAILED DESCRIPTION OF PARTICULAR EMBODIMENTS

The examples that follow propose an example of preparation of a copolymer that may be used as tensioning agent in accordance with the invention, and also various formulations comprising this copolymer.

### Example 1

This example illustrates the preparation of a triblock copolymer comprising:
a first block comprising units derived from styrene with a number-average molecular mass of 30 000 g/mol;
a second block consisting of units derived from ethyl acrylate with a number-average molecular mass of 10 000 g/mol;
a third block comprising units derived from styrene with a number-average molecular mass of 30 000 g/mol.

The procedure is based on a process that may be broken down into three distinct phases, a first step which is the production of a polystyrene block, a second step which is the synthesis of a poly(ethyl acrylate) block after the first block, and a third step which is the synthesis of a polystyrene block after the second block, to obtain the polystyrene-b-poly(ethyl acrylate)-b-polystyrene triblock.

The synthesis of this copolymer is performed in a 2-litre glass reactor of SVL type. The maximum working charge of this type of reactor is 1.5 litres. The internal temperature of the reactor is regulated with a cryostat of Huber type. The temperature is measured using a pt 100 probe dipped into the reactor and serving for regulation. The stirring unit is a stainless-steel paddle. The spin speed of the spindle is about 200 rpm. The reactor is also equipped with a reflux device (coil condenser) that is efficient enough to allow reflux of the monomers without loss of product.

The process performed is an emulsion polymerization process in water, of latex type.

### Step 1: Preparation of the First Block.

This first step consists in preparing the first block, which consists of a statistical copolymer of styrene and of methacrylic acid, with a styrene/methacrylic acid mass ratio=98/2 of targeted theoretical mass: Mₙ=30 000 g/mol.

568.0 g of water, 12.0 g of sodium dodecylsulfate and 0.95 g of sodium carbonate Na₂CO₃ are introduced, at room temperature, as the feedstock. The mixture obtained is stirred for 30 minutes (200 rpm) under nitrogen. The temperature is then raised to 75° C. and a mixture 1 is then added, comprising:
25.71 g of styrene (St),
0.510 g of methacrylic acid (MAA), and
1.790 g of xanthate (CH₃) (CO₂CH₃)CH—S(C═S)OCH₂CH₃.

The mixture is brought to 85° C. and a solution of 0.390 g of ammonium persulfate (NH₄)₂S₂O₈ dissolved in 10.0 g of water is then introduced.

After 5 minutes, the addition of a mixture 2 is started, comprising:
231.4 g of styrene (St) and
4.60 g of methacrylic acid (MAA).

The addition is continued for 115 minutes. After complete addition of the various ingredients, the copolymer emulsion obtained is maintained at 85° C. for two hours.

A sample (˜5 g) is then taken and analysed by steric exclusion chromatography (SEC) in THF. Its measured number-average molecular mass Mn is equal to 26 600 g/mol as polystyrene equivalents (calibration with linear polystyrene standards). Its polydispersity index Mw/Mn is equal to 2.0.

An analysis of the sample by gas chromatography reveals that the conversion of the monomers is greater than 99%.

### Step 2: Preparation of the Second Block.

This second step consists of the synthesis of an ethyl acrylate polymer.

The emulsion copolymer obtained above in step 1 is used as starting material, after having removed ˜5 g for analysis, and without stopping the heating.

0.390 g of ammonium persulfate (NH₄)₂S₂O₈ diluted in 50.0 g of water is introduced continuously over one hour.

The following are then simultaneously added over one hour at 85° C.:
85.7 g of ethyl acrylate (EA).

The system is maintained at this temperature for a further two hours.

A sample (˜5 g) is then taken and analysed by steric exclusion chromatography (SEC) in THF. Its measured number-average molecular mass Mn is equal to 37 000 g/mol as polystyrene equivalents (calibration with linear polystyrene standards). Its polydispersity index Mw/Mn is equal to 1.9.

An analysis of the sample by gas chromatography reveals that the conversion of the monomers is greater than 99%.

### Step 3: Preparation of the Third Block

This first step consists in preparing the third block, which consists of a statistical copolymer of styrene and of methacrylic acid, with a styrene/methacrylic acid mass ratio=98/2, of targeted theoretical mass: Mₙ=30 000 g/mol.

The emulsion copolymer obtained above in step 2 is used as starting material, after having removed ˜5 g for analysis, and without stopping the heating.

0.390 g of ammonium persulfate (NH₄)₂S₂O₈ diluted in 50.0 g of water is introduced continuously over three hours. Next, a mixture 3 is added over three hours at 85° C., comprising:
50.0 g of water,
0.95 g of sodium carbonate Na₂CO₃.

Simultaneously, a mixture 4 is added, comprising:
257.1 g of styrene (St), and
5.14 g of methacrylic acid (MAA).

After complete addition of the various ingredients, the copolymer emulsion obtained is maintained at 85° C. for one hour. 1.20 g of tert-butylbenzyl peroxide are then introduced in a single portion and the addition of a mixture 5 is commenced, comprising:
0.600 g of erythorbic acid,
20.0 g of water.

The addition is continued for 60 minutes. After complete addition of the various ingredients, the emulsion is cooled to ˜25° C. over one hour.

A sample (˜5 g) is then taken and analysed by steric exclusion chromatography (SEC) in THF. Its measured number-average molecular mass Mn is equal to 56 800 g/mol as polystyrene equivalents (calibration with linear polystyrene standards). Its polydispersity index Mw/Mn is equal to 1.9.

An analysis of the sample by gas chromatography reveals that the conversion of the monomers is greater than 99.8%.

The product obtained is a dispersion in water of the copolymer (latex), with a solids content of about 44%.

### Example 2

### Preparation of a Polystyrene-Block-Poly(Ethyl acrylate)-block-polysytrene Triblock Copolymer “pS₂₅ₖ-pEA₂₀ₖ-pS₂₅ₖ”

The procedure is based on a process that may be broken down into three distinct phases, a first step which is the production of a polystyrene block, a second step which is the synthesis of a poly(ethyl acrylate) block after the first block, and a third step which is the synthesis of a polystyrene block after the second block, to obtain the polystyrene-b-poly(ethyl acrylate)-b-polystyrene triblock.

The synthesis of this copolymer is performed in a 2-litre glass reactor of SVL type. The maximum working charge of this type of reactor is 1.5 litres. The internal temperature of the reactor is regulated with a cryostat of Huber type. The temperature is measured using a pt 100 probe dipped into the reactor and serving for regulation. The stirring unit is a stainless-steel paddle. The spin speed of the spindle is about 200 rpm. The reactor is also equipped with a reflux device (coil condenser) that is efficient enough to allow reflux of the monomers without loss of product.

The process performed is an emulsion polymerization process in water, of latex type.

Step 1: Preparation of a First Polystyrene Block with a Theoretical Molecular Mass of about 25 000 g/mol “pS₂₅ₖ”

In reality, this is a synthesis of a statistical copolymer of styrene and of methacrylic acid, with an St/MAA mass ratio=98/2. Targeted theoretical mass: Mₙ=25 000 g/mol. This will be referred to as a polystyrene block for the sake of simplicity of the terminology.

518.2 g of water, 6.250 g of sodium dodecylsulfate and 0.714 g of sodium carbonate Na₂CO₃ are introduced, at room temperature as the feedstock. The mixture obtained is stirred for 30 minutes (200 rpm) under nitrogen. The temperature is then raised to 75° C. and a mixture 1 is then added, comprising:
17.86 g of styrene (St),
0.357 g of methacrylic acid (MAA), and
1.486 g of xanthate (CH₃) (CO₂CH₃)CH—S(C═S)OCH₂CH₃.

The mixture is brought to 85° C. and a solution of 0.085 g of sodium persulfate Na₂S₂O₈ dissolved in 1.70 g of water is then introduced.

After 5 minutes, the addition of a mixture 2 is started, comprising:
160.7 g of styrene (St) and
3.21 g of methacrylic acid (MAA).

Simultaneously, the addition of a mixture 3 is started, comprising 0.255 g of sodium persulfate Na₂S₂O₈ dissolved in 5.10 g of water.

The addition is continued for 90 minutes. After complete addition of the various ingredients, the copolymer emulsion obtained is maintained at 85° C. for two hours.

A sample (˜5 g) is then taken and analysed by steric exclusion chromatography (SEC) in THF. Its measured number-average molecular mass Mn is equal to 22 000 g/mol as polystyrene equivalents (calibration with linear polystyrene standards). Its polydispersity index Mw/Mn is equal to 2.2.

An analysis of the sample by gas chromatography reveals that the conversion of the monomers is greater than 99%.

Step 2: Preparation of a Second Block of poly(ethyl acrylate) with a Theoretical Molecular Mass of about 10 000 g/mol to Obtain a polystyrene-block-poly(ethyl acrylate) Diblock Copolymer, “pS₂₅ₖ-pEA₂₀ₖ”

The emulsion copolymer obtained above in step 1 is used as starting material, after having removed ˜5 g for analysis, and without stopping the heating.

0.170 g of sodium persulfate Na₂S₂O₈ diluted in 3.4 g of water is introduced continuously over 90 minutes.

The following are then simultaneously added over 90 minutes at 85° C.:
142.9 g of ethyl acrylate (EA).

The system is maintained at this temperature for a further two hours.

A sample (˜5 g) is then taken and analysed by steric exclusion chromatography (SEC) in THF. Its measured number-average molecular mass Mn is equal to 32 000 g/mol as polystyrene equivalents (calibration with linear polystyrene standards). Its polydispersity index Mw/Mn is equal to 2.6.

An analysis of the sample by gas chromatography reveals that the conversion of the monomers is greater than 99%.

Step 3: Preparation of a Third Block of Polystyrene with a Theoretical Molecular Mass of about 25 000 g/mol to Obtain a polystyrene-block-poly(ethyl acrylate)-block-polystyrene Triblock Copolymer “pS₂₅ₖ-pEA₂₀ₖ-pS₂₅ₖ”

In reality, this is a synthesis of a statistical copolymer of styrene and of methacrylic acid. St/MAA mass ratio=98/2. Targeted theoretical mass: Mₙ=25 000 g/mol. This will be referred to as a polystyrene block for the sake of simplicity of the terminology.

The emulsion copolymer obtained above in step 2 is used as starting material, after having removed ˜5 g for analysis, and without stopping the heating.

0.340 g of sodium persulfate Na₂S₂O₈ diluted in 6.8 g of water is introduced continuously over two hours. Simultaneously, a mixture 4 is added over two hours at 85° C., comprising:
97.90 g of water,
1.146 g of sodium carbonate Na₂CO₃,
3.75 g of sodium dodecylsulfate.

Simultaneously, a mixture 5 is added, comprising:
178.6 g of styrene (St), and
3.57 g of methacrylic acid (MAA).

After complete addition of the various ingredients, the copolymer emulsion obtained is maintained at 85° C. for two hours.

0.500 g of tert-butylbenzyl peroxide is then introduced in a single portion and the addition of a mixture 6 is commenced, comprising:
0.250 g of erythorbic acid,
5.0 g of water.

The addition is continued for 60 minutes. After complete addition of the various ingredients, the emulsion is cooled to ˜25° C. over one hour.

A sample (˜5 g) is then taken and analysed by steric exclusion chromatography (SEC) in THF. Its measured number-average molecular mass Mn is equal to 40 000 g/mol as polystyrene equivalents (calibration with linear polystyrene standards). Its polydispersity index Mw/Mn is equal to 2.9.

An analysis of the sample by gas chromatography reveals that the conversion of the monomers is greater than 99.8%.

The product obtained is a dispersion in water of the copolymer (latex), with a solids content of about 45%.

### Example 3

### Preparation of a Polystyrene-Block-poly(ethyl acrylate)-block-polystyrene Triblock Copolymer “pS_{32.5k}-pEA₅ₖ-pS_{32.5k}”

The procedure is based on a process that may be broken down into three distinct phases, a first step which is the production of a polystyrene block, a second step which is the synthesis of a poly(ethyl acrylate) block after the first block, and a third step which is the synthesis of a polystyrene block after the second block, to obtain the polystyrene-b-poly(ethyl acrylate)-b-polystyrene triblock.

The synthesis of this copolymer is performed in a 2-litre glass reactor of SVL type. The maximum working charge of this type of reactor is 1.5 litres. The internal temperature of the reactor is regulated with a cryostat of Huber type. The temperature is measured using a pt 100 probe dipped into the reactor and serving for regulation. The stirring unit is a stainless-steel paddle. The spin speed of the spindle is about 200 rpm. The reactor is also equipped with a reflux device (coil condenser) that is efficient enough to allow reflux of the monomers without loss of product.

The process performed is an emulsion polymerization process in water, of latex type.

Step 1: Preparation of a First Block of Polystyrene with a Theoretical Molecular Mass of about 32 500 g/mol “pS_{32.5k}”

In reality, this is a synthesis of a statistical copolymer of styrene and of methacrylic acid, with an St/MAA mass ratio=98/2. Targeted theoretical mass: Mₙ=32 500 g/mol. This will be referred to as a polystyrene block for the sake of simplicity of the terminology.

515.0 g of water, 6.250 g of sodium dodecylsulfate and 0.929 g of sodium carbonate Na₂CO₃ are introduced, at room temperature as the feedstock. The mixture obtained is stirred for 30 minutes (200 rpm) under nitrogen. The temperature is then raised to 75° C. and a mixture 1 is then added, comprising:
23.21 g of styrene (St),
0.464 g of methacrylic acid (MAA), and
1.486 g of xanthate (CH₃) (CO₂CH₃)CH—S(C═S)OCH₂CH₃.

The mixture is brought to 85° C. and a solution of 0.085 g of sodium persulfate Na₂S₂O₈ dissolved in 1.70 g of water is then introduced.

After 5 minutes, the addition of a mixture 2 is started, comprising:
208.9 g of styrene (St) and
4.18 g of methacrylic acid (MAA).

Simultaneously, the addition of a mixture 3 is started, comprising 0.255 g of sodium persulfate Na₂S₂O₈ dissolved in 5.10 g of water.

The addition is continued for 80 minutes. After complete addition of the various ingredients, the copolymer emulsion obtained is maintained at 85° C. for two hours.

A sample (˜5 g) is then taken and analysed by steric exclusion chromatography (SEC) in THF. Its measured number-average molecular mass Mn is equal to 29 000 g/mol as polystyrene equivalents (calibration with linear polystyrene standards). Its polydispersity index Mw/Mn is equal to 2.2.

An analysis of the sample by gas chromatography reveals that the conversion of the monomers is greater than 99%.

Step 2: Preparation of a Second Block of poly(ethyl acrylate) with a Theoretical Molecular Mass of about 10 000 g/mol to Obtain a polystyrene-block-poly(ethyl acrylate) Diblock Copolymer, “pS_{32.5k}-pEA₅ₖ”

The emulsion copolymer obtained above in step 1 is used as starting material, after having removed ˜5 g for analysis, and without stopping the heating.

0.170 g of sodium persulfate Na₂S₂O₈ diluted in 3.4 g of water is introduced continuously over 90 minutes.

The following are then simultaneously added over 90 minutes at 85° C.:
35.7 g of ethyl acrylate (EA).

The system is maintained at this temperature for a further two hours.

A sample (˜5 g) is then taken and analysed by steric exclusion chromatography (SEC) in THF. Its measured number-average molecular mass Mn is equal to 34 000 g/mol as polystyrene equivalents (calibration with linear polystyrene standards). Its polydispersity index Mw/Mn is equal to 2.3.

An analysis of the sample by gas chromatography reveals that the conversion of the monomers is greater than 99%.

Step 3: Preparation of a Third Block of Polystyrene with a Theoretical Molecular Mass of about 32 500 g/mol to Obtain a polystyrene-block-poly(ethyl acrylate)-block-polystyrene Triblock Copolymer “pS_{35.5k}-pEA₅ₖ-pS_{32.5k}”

In reality, this is a synthesis of a statistical copolymer of styrene and of methacrylic acid. St/MAA mass ratio=98/2. Targeted theoretical mass: Mₙ=32 500 g/mol. This will be referred to as a polystyrene block for the sake of simplicity of the terminology.

The emulsion copolymer obtained above in step 2 is used as starting material, after having removed ˜5 g for analysis, and without stopping the heating.

0.340 g of sodium persulfate Na₂S₂O₈ diluted in 6.8 g of water is introduced continuously over two hours. Simultaneously, a mixture 4 is added over two hours at 85° C., comprising:
104.4 g of water,
1.468 g of sodium carbonate Na₂CO₃,
3.75 g of sodium dodecylsulfate.

Simultaneously, a mixture 5 is added, comprising:
1232.1 g of styrene (St), and
4.64 g of methacrylic acid (MAA).

After complete addition of the various ingredients, the copolymer emulsion obtained is maintained at 85° C. for two hours.

0.500 g of tert-butylbenzyl peroxide is then introduced in a single portion and the addition of a mixture 6 is commenced, comprising:
0.250 g of erythorbic acid,
5.0 g of water.

The addition is continued for 60 minutes. After complete addition of the various ingredients, the emulsion is cooled to ˜25° C. over one hour.

A sample (˜5 g) is then taken and analysed by steric exclusion chromatography (SEC) in THF. Its measured number-average molecular mass Mn is equal to 50 000 g/mol as polystyrene equivalents (calibration with linear polystyrene standards). Its polydispersity index Mw/Mn is equal to 2.6.

An analysis of the sample by gas chromatography reveals that the conversion of the monomers is greater than 99.8%.

The product obtained is a dispersion in water of the copolymer (latex), with a solids content of about 45%.

### Example 4

### Cosmetic Composition in Serum Form

*Phase A

| | |
|---|---|
| Water | 80.05 g |
| Methyl vinyl ether/maleic | 0.20 g |
| anhydride copolymer | |
| (Stabilize QM from ISP) | |
| Xanthan gum | 0.20 g |
| Methyl p-hydroxybenzoate | 0.20 g |
| Phenoxyethanol | 0.35 g |

*Phase B

| | |
|---|---|
| Triethanolamine | 0.20 g |
| Water | 1 g |

* Phase C

| | |
|---|---|
| Polyacrylamide and C₁₃-C₁₄ | 1.00 g |
| isoparaffin and Laureth-7 | |
| (Sepigel 305 from SEPPIC) | |
| Diazolidinylurea | 0.30 g |

### *Phase D

Emulsion of triblock copolymer prepared according to Example 1: 16.50 g containing 42.6% active material.

### Procedure

Phase A is heated to about 75° C. with stirring, and phase B is then poured into phase A. Next, the heating is stopped and stirring is continued until the mixture has returned to room temperature, and phases C and D are then added. Gentle stirring is then continued for 30 minutes.

This composition was tested on a panel of women from 40 to 60 years old bearing wrinkles and fine lines around the eyes. After applying this composition, a mechanical smoothing effect on the wrinkles and fine lines was observed. Furthermore, this composition has the effect of reducing the visibility of the skin's microrelief, giving a “soft-focus” veil effect that makes the grain of the skin finer. It was not found that an unattractive lacquered film appeared with this composition.

### Example 5

### Cosmetic Composition (Essence)

*Phase A

| | |
|---|---|
| Cyclohexasiloxane | 5 g |
| Hydrogenated polyisobutene | 5 g |
| Dimethicone PEG-7 | 2 g |
| phosphate | |

*Phase B

| | |
|---|---|
| Xanthan gum | 0.4 g |
| Sodium hydroxide | 0.3 g |
| Isobutyl p-hydroxybenzoate | 0.021 g |
| Water | 69.15 g |
| Carboxypolymethylene | 0.4 g |
| Acrylic copolymer (Pemulen | 0.25 g |
| TR1 from Noveon) | |
| Propyl p-hydroxybenzoate | 0.021 g |
| Butyl p-hydroxybenzoate | 0.042 g |
| Methyl p-hydroxybenzoate | 0.174 g |
| Ethyl p-hydroxybenzoate | 0.042 g |
| Phenoxyethanol | 0.7 g |

### *Phase C.

Emulsion of triblock copolymer prepared according to Example 1: 16.50 g containing 42.6% active material.

### *Procedure

Phase B is heated to about 75° C. with stirring until a homogeneous gel is obtained.

Phase A is heated to about 75° C. An emulsion is prepared by incorporating phase A into phase B.

Phase C is incorporated into the emulsion obtained above at a temperature of 40-45° C. and stirring is continued until the emulsion has completely cooled.

This composition was tested on a panel of women from 40 to 60 years old bearing wrinkles and fine lines around the eyes. After applying this composition, a mechanical smoothing effect on the wrinkles and fine lines was observed. Furthermore, this composition has the effect of reducing the visibility of the skin's microrelief, giving a “soft-focus” veil effect that makes the grain of the skin finer. No appearance of an unattractive lacquered film was observed with this composition.

### Comparative Example 5

For comparative purposes, a diblock copolymer comprising:
a first block consisting of units derived from styrene with a number-average molecular mass of 5000 g/mol;
a second block consisting of units derived from ethyl acrylate with a number-average molecular mass of 60 000 g/mol,
   in which the ratio between the units derived from styrene and the units derived from ethyl acrylate is less than 1, was tested to determine its tensioning effect. It was found that this copolymer had no tensioning effect.

### Example 6

A composition with a skin-tensioning effect is prepared, comprising the following ingredients:

| Mixture of (C14-C15)dialkyl tartrate, | 1.5 | g |
|---|---|---|
| cetylstearyl alcohol and oxyethylenated | | |
| (25 EO) oxypropylenated (25 PO) lauryl | | |
| alcohol | | |
| (Cosmacol PSE from the company Sasol) | | |
| Mixture of glyceryl mono/distearate and of | 2 | g |
| polyethylene glycol stearate (100 EO) | | |
| (Arlacel 165FL from Uniqema) | | |
| Stearyl alcohol | 1 | g |
| Cyclohexasiloxane | 10 | g |
| Aqueous emulsion of triblock copolymer of | 15.3 | g |
| Example 2 | | |
| Polyacrylamidomethylpropanesulfonic acid | 0.4 | g |
| partially neutralized with ammonia and | | |
| crosslinked | | |
| (Hostacerin AMPS from Clariant) | | |
| Xanthan gum | 0.2 | g |
| Disodium salt of ethylenediaminetetra- | 0.05 | g |
| acetic acid | | |
| Preserving agents | qs | |
| Water | qs 100 | g |

### Example 6

A composition with a skin-tensioning effect is prepared, comprising the following ingredients:

| Dimethicone copolyol phosphate | 2 | g |
|---|---|---|
| (Pecosil PS 100 from Phoenix Chemical) | | |
| Cyclohexasiloxane | 5 | g |
| Hydrogenated isoparaffin | 5 | g |
| (Parleam oil from NOF) | | |
| Carboxyvinyl polymer | 0.4 | g |
| (Carbopol 980 from Noveon) | | |
| Sodium hydroxide | 0.3 | g |
| Xanthan gum | 0.4 | g |
| Acrylic acid/(C10-C30)alkyl acrylate | 0.25 | g |
| copolymer | | |
| (Pemulen TR2 from Noveon) | | |
| Aqueous emulsion of triblock copolymer of | 15.3 | g |
| Example 3 | | |
| Preserving agents | qs | |
| Water | qs 100 | g |

1. A method of using a cosmetic, as a skin-tensioning agent in a cosmetic composition, of a copolymer comprising units derived from styrene and units derived from ethyl (meth)acrylate, in which the weight ratio between the units derived from styrene and the units derived from ethyl (meth)acrylate is greater than or equal to 1.

2. The method according to claim 1, in which the cosmetic composition is an anti-wrinkle composition.

3. The method according to claim 1, in which the weight ratio between the units derived from styrene and the units derived from ethyl (meth)acrylate is greater than 2.

4. The method according to claim 1, in which the weight ratio between the units derived from styrene and the units derived from ethyl (meth)acrylate is greater than 5.

5. The method according to claim 1, in which the weight ratio between the units derived from styrene and the units derived from ethyl (meth)acrylate is greater than 20.

6. The method according to claim 1, in which the copolymer is a random copolymer.

7. The method according to claim 1, in which the copolymer is a block copolymer.

8. The method according to claim 7, in which the block copolymer is a linear block copolymer of the type A-[B-A]ₙ or B-[A-B]ₙ or [A-B]ₙ in which A is a block comprising at least 50% by weight of units derived from styrene, B is a block comprising at least 50% by weight of units derived from ethyl (meth)acrylate, and n is a number greater than or equal to 1.

9. The method according to claim 8, in which the block copolymer is a triblock copolymer.

10. The method according to claim 9, in which the triblock copolymer comprises:
a first block comprising units derived from styrene with a number-average molecular mass of 30 000 g/mol;
a second block consisting of units derived from ethyl acrylate with a number-average molecular mass of 10 000 g/mol;
a third block comprising units derived from styrene with a number-average molecular mass of 30 000 g/mol.

11. The method according to claim 1, in which the number-average molecular mass of the overall copolymer is greater than 10 000 g/mol.

12. The method according to claim 1, in which the composition is suitable for topical application to facial skin.

13. Cosmetic process for reducing the wrinkles of wrinkled skin, comprising a step that consists in applying to the said skin a composition comprising, in a physiologically acceptable medium, at least one copolymer as defined according to claim 1.

14. Cosmetic treatment process according to claim 13, in which the application is made to the contour of the eyes.
